(19) United States
(12) Patent Application Publication
(10) Pub. No.: US 2008/0270041 A1
(43) Pub. Date: Oct. 30, 2008

(54) SYSTEM AND METHOD FOR BROAD-BASED MULTIPLE SCLEROSIS ASSOCIATION GENE TRANSCRIPT TEST

(75) Inventors: Dean I. Sproles, Seattle, WA (US); Roy L. Swank, Portland, OR (US)

Correspondence Address: DAVIS WRIGHT TREMAINE, LLP/Seattle, 1201 Third Avenue, Suite 2200, SEATTLE, WA, 98101-3045, US

(73) Assignee: IGD INTEL LLC, Seattle, WA (US)

(21) Appl. No.: 11/937,896

(22) Filed: Nov. 9, 2007

Related U.S. Application Data
(60) Provisional application No. 60/913,758, filed on Apr. 24, 2007.

(51) Int. Cl. G01N 33/48; G06F 19/00

(52) U.S. Cl. 702/20

(57) ABSTRACT

Broad-based gene association transcript test for multiple sclerosis and data structure. Multiple sclerosis considerations for this unique test include a custom set of genetic sequences associated in peer-reviewed literature with various known multiple sclerosis related to exposure to toxic substances. Such multiple sclerosis symptoms include specific genetic expressions linked to symptoms of the disease. The base dataset may be developed through clinical samples obtained by third-parties. Online access of real-time phenotype/genotype associative testing for physicians and patients may be promoted through an analysis of a customized microarray testing service.

### CROSS-REFERENCE TO PROVISIONAL PATENT APPLICATION

This patent application claims priority from a related provisional patent application entitled ‘BROAD-BASED MULTIPLE SCLEROSIS ASSOCIATION GENE TRANSCRIPT TEST’ filed on Apr. 24, 2007 which is incorporated herein in its entirety.

### BACKGROUND

Multiple Sclerosis (commonly referred to as MS) is a chronic, inflammatory, demyelinating disease that affects the central nervous system of a person. MS exhibits a variety of symptoms or traits, including changes in sensation, visual problems, muscle weakness, depression, difficulties with coordination and speech, severe fatigue, cognitive impairment, problems with balance, overheating, and in more severe cases, even impaired mobility and complete disability.

Multiple sclerosis affects neurons which are cells of the brain and spinal cord that carry information, create thought and perception, and allow the brain to control the body. Surrounding and protecting some of these neurons is a fatty layer known as the myelin sheath, which helps neurons carry electrical signals. MS causes gradual destruction of myelin (demyelination) and transection of neuron axons in patches throughout the brain and spinal cord. The name multiple sclerosis refers to the multiple scars (or scleroses) on the myelin sheaths. This scarring causes symptoms which vary widely depending upon which signals are interrupted.

Multiple sclerosis may take several different forms, with new symptoms occurring either in discrete attacks or slowly accruing over time. Between attacks, symptoms may resolve completely, but permanent neurologic problems often persist, especially as the disease advances. MS currently does not have a cure, though several treatments are available that may slow the appearance of new symptoms.

The Swank Multiple Sclerosis Foundation is a public charity that provides information and resources on the Swank Low Fat Diet, vitamin supplements, and life-style changes beneficial to patients with Multiple Sclerosis, as well as their families and friends, as pioneered by Roy L. Swank, M.D., Ph.D. The foundation has become an authority with regard to association with resources, information, research, and treatment surrounding Multiple Sclerosis. The Vision of the Foundation is to increase awareness and expand implementation of the successful holistic treatment of Multiple Sclerosis centered around 50 years of monitored clinical studies of individuals following the Swank Low Fat Diet. Further, the foundation fosters contact between newly diagnosed patients and those who have followed the treatment over a long period of time. Sharing of information regarding recipes supplied by patients and friends through the website and related publications is encouraged as well as through a message board and chat-room which act as a contact source and focus of self-help for patients and other interested parties. As a supplement and as the result of numerous clinical studies conducted worldwide, much knowledge and information has been collected about the genetic makeup of those affected by multiple sclerosis.

As with many diseases, genealogy plays a significant role in a person's development and susceptibility to multiple sclerosis. That is, a person diagnosed with MS will exhibit specific genetic expressions that are typically common between all persons who are diagnosed with MS. Although not consistent from person to person, certainly evidence shows that a number of gene expressions are related to MS.

A person's genetic makeup is reflected through Deoxyribonucleic Acids (DNA). DNA is a molecule that is comprised of sequences of nucleic acids that form the code which contains the genetic instructions for the development and functioning of living organisms. A DNA sequence or genetic sequence is a succession of any of four specific nucleic acids representing the primary structure of a real or hypothetical DNA molecule or strand, with the capacity to carry information. As is well understood in the art, the possible nucleic acids (letters) are A, C, G, and T, representing the four nucleotide subunits of a DNA strand—adenine, cytosine, guanine, and thymine bases covalently linked to phospho-backbone. Typically the sequences are printed abutting one another without gaps, as in the sequence AAAGTCTGAC. A succession of any number of nucleotides greater than four may be called a sequence. With regard to its biological function, which may depend on context, a sequence may be sense or anti-sense, and either coding or non-coding.

Ribonucleic acid (RNA) is a nucleic acid polymer consisting of nucleotide monomers, that acts as a messenger between DNA and ribosomes, and that is also responsible for making proteins by coding for amino acids. RNA polynucleotides contain ribose sugars unlike DNA, which contains deoxyribose; and RNA substitutes for uracil in any position of DNA where thymine is present. RNA is transcribed (synthesized) from DNA by enzymes called RNA polymerases and further processed by other enzymes. RNA serves as the template for translation of genes into proteins, transferring amino acids to the ribosome to form proteins, and also translating the transcript into proteins.

As previously mentioned, certain genetic disorders may result from DNA sequences being incorrectly coded. A Single Nucleotide Polymorphism or S.N.P. (often time called a “snip”) is a DNA sequence variation occurring when a single nucleotide—A, T, C, or G—in the genome (or other shared sequence) differs between members of a species (or between paired chromosomes in an individual). For example, two sequenced DNA fragments from different individuals, AAGCCTA to AAGCTTA, contain a difference in a single nucleotide. In this case we say that there are two alleles: C and T. High degrees of variation within coding and non-coding regions exist and are the topic of ongoing research efforts.

Within a population, Single Nucleotide Polymorphisms can be assigned a minor allele frequency—the ratio of chromosomes in the population carrying the less common variant to those with the more common variant. Usually one will want to refer to Single Nucleotide Polymorphisms with a minor allele frequency of ≧ 1% (or 0.5% etc.), rather than to “all Single Nucleotide Polymorphisms” (a set so large as to be unwieldy). It is important to note that there are variations between human populations, so a Single Nucleotide Polymorphism that is common enough for inclusion in one geographical or ethnic group may be much rarer in another. This same concept may be applied across demographic groups and regions with respect to multiple sclerosis as well.

Single Nucleotide Polymorphisms may fall within coding sequences of genes, noncoding regions of genes, or in the intergenic regions between genes. Single Nucleotide Polymorphisms within a coding sequence will not necessarily change the amino acid sequence of the protein that is produced, due to degeneracy of the genetic code. A Single Nucleotide Polymorphism in which both forms lead to the same polypeptide sequence is termed synonymous (sometimes called a silent mutation)—if a different polypeptide sequence is produced they are non-synonymous. Single Nucleotide Polymorphisms that are not in protein coding regions may still have consequences for gene splicing, transcription factor binding, or the sequence of non-coding RNA.

Variations in the DNA sequences of humans can affect how humans develop diseases, respond to pathogens, chemicals, drugs, etc. However, their greatest importance in biomedical research is for comparing regions of the genome that between cohorts (such as with matched cohorts with and without a disease). Technologies from Affymetrix™ and Illumina™ allow for genotyping hundreds of thousands of Single Nucleotide Polymorphisms for typically under $1,000.00 in a couple of days.

A gene is a segment of nucleic acid that contains the information necessary to produce a functional product, usually a protein. Genes contain regulatory regions dictating under what conditions the product is produced, transcribed regions dictating the structure of the product, and/or other functional sequence regions. Genes interact with each other to influence physical development and behavior. Genes consist of a long strand of DNA (RNA in some viruses) that contains a promoter, which controls the activity of a gene, and a coding sequence, which determines what the gene produces. When a gene is active, the coding sequence is copied in a process called transcription, producing an RNA copy of the gene's information. This RNA can then direct the synthesis of proteins via the genetic code. However, RNAs can also be used directly, for example as part of the ribosome. These molecules resulting from gene expression, whether RNA or protein, are known as gene products.

The total complement of genes in an organism or cell is known as its genome. The genome size of an organism is loosely dependent on its complexity. The number of genes in the human genome is estimated to be just under 3 billion base pairs and about 30,000 genes.

Microarray analysis techniques are typically used in interpreting the data generated from experiments on DNA, RNA, and protein microarrays, which allow researchers to investigate the expression state of a large number of genes—in many cases, an organism's entire genome—in a single experiment. Such experiments generate a very large volume of genetic data that can be difficult to analyze, especially in the absence of good gene annotation. Most microarray manufacturers, such as Affymetrix™, provide commercial data analysis software with microarray equipment such as plate readers.

Specialized software tools for statistical analysis to determine the extent of over- or under-expression of a gene in a microarray experiment relative to a reference state have also been developed to aid in identifying genes or gene sets associated with particular phenotypes. Such statistics packages typically offer the user information on the genes or gene sets of interest, including links to entries in databases such as NCBI's GenBank™ and curated databases such as Biocarta™ and Gene Ontology.

As a result of a statistical analysis, specific aspects of an organism may be genotyped. Genotyping refers to the process of determining the genotype of an individual with a biological assay, e.g., multiple sclerosis and various MS-traits. Current methods of doing this include Polymerase Chain Reaction (PCR), DNA sequencing, and hybridization to DNA microarrays or beads. The technology is intrinsic for tests on father-/motherhood and in clinical research for the investigation of multiple sclerosis-associated genes.

Further, phenotyping is also a known process for assessing phenotypes. The phenotype of an individual organism is either its total physical appearance and constitution or a specific manifestation of a trait, such as size, eye color, or behavior that varies between individuals. Phenotype is determined to a large extent by genotype, or by the identity of the alleles that an individual carries at one or more positions on the chromosomes. Many phenotypes are determined by multiple genes and influenced by environmental factors. Thus, the identity of one or a few known alleles does not always enable prediction of the phenotype.

However, this genotyping process is typically accomplished for a single patient or research sample in a single sampling for a single iteration. As such, the results are relatively isolated with respect to any possible comparison and analysis of other similarly situated patients with Multiple Sclerosis. Furthermore, such isolation leads to inefficiencies in diagnostics and treatment of the underlying results of the test. Without a system for allowing the sharing of underlying data, all potential benefits of aggregating the data are lost. What is needed is a broad-based multiple sclerosis diagnostic and treatment suggestive gene transcription test capable of allowing the assimilation of a wide range of associated genomic data from a wide range of individuals who currently have or may be diagnosed with multiple sclerosis.

### BRIEF DESCRIPTION OF THE DRAWINGS

The foregoing aspects and many of the attendant advantages of the claims will become more readily appreciated as the same become better understood by reference to the following detailed description, when taken in conjunction with the accompanying drawings, wherein:

FIG. 1 shows a diagram of a method for preparing a microarray to be used in a broad-based gene association transcript test for multiple sclerosis according to an embodiment of an invention disclosed herein;

FIG. 2 shows a diagrammatic representation of a method for collecting genetic material samples from several sources and detecting and isolating strands of genetic material for grouping according to an embodiment of an invention disclosed herein;

FIG. 3 is a diagrammatic representation of a suitable computing environment in which some aspects of a broad-based gene association transcript test for multiple sclerosis may be practiced according to an embodiment of an invention disclosed herein;

FIG. 4 is a diagrammatic representation of a networked computing environment in which some aspects of a broad-based gene association transcript test for multiple sclerosis may be practiced according to an embodiment of an invention disclosed herein;

FIG. 5 shows a typical arrangement of data that may be associated in a database of information derived from a broad-based gene association transcript test for multiple sclerosis according to an embodiment of an invention disclosed herein;

FIG. 6 is a flow chart of a method for collecting genetic material and preparing the collection for assessment in a broad-based gene association transcript test for multiple sclerosis according to an embodiment of an invention disclosed herein; and

FIG. 7 is a flow chart of a method for assessing collected data from a broad-based gene association transcript test for multiple sclerosis according to an embodiment of an invention disclosed herein.

### DETAILED DESCRIPTION

The following discussion is presented to enable a person skilled in the art to make and use the subject matter disclosed herein. The general principles described herein may be applied to embodiments and applications other than those detailed above without departing from the spirit and scope of the present detailed description. The present disclosure is not intended to be limited to the embodiments shown, but is to be accorded the widest scope consistent with the principles and features disclosed or suggested herein.

The subject matter disclosed herein is related to transcriptional detection of single nucleotide polymorphisms (SNP) and insertion/deletion (I/D) genetic polymorphisms through a proportional analysis of RNA sequences detected through fluorescence hybridization on a custom manufactured microarray gene expression platform. Specifically, such SNPs that are related to multiple sclerosis (MS) are of interest in this gene transcript test as specific gene expressions are typically synonymous with specific genetic disorders and traits of MS. SNPs may be identified through a specific design method (as SNPs are typically assessed through DNA analysis) associated with a genotyping system and method for multiple sclerosis. A base data set may be used to further assist in the analysis. The base dataset may be developed through clinical samples obtained by third-parties clinical groups, and in partial association with various communities such as the Swank Multiple Sclerosis Foundation. Further, online access of real-time phenotype/genotype associative testing for physicians and patients may be promoted through a testing service.

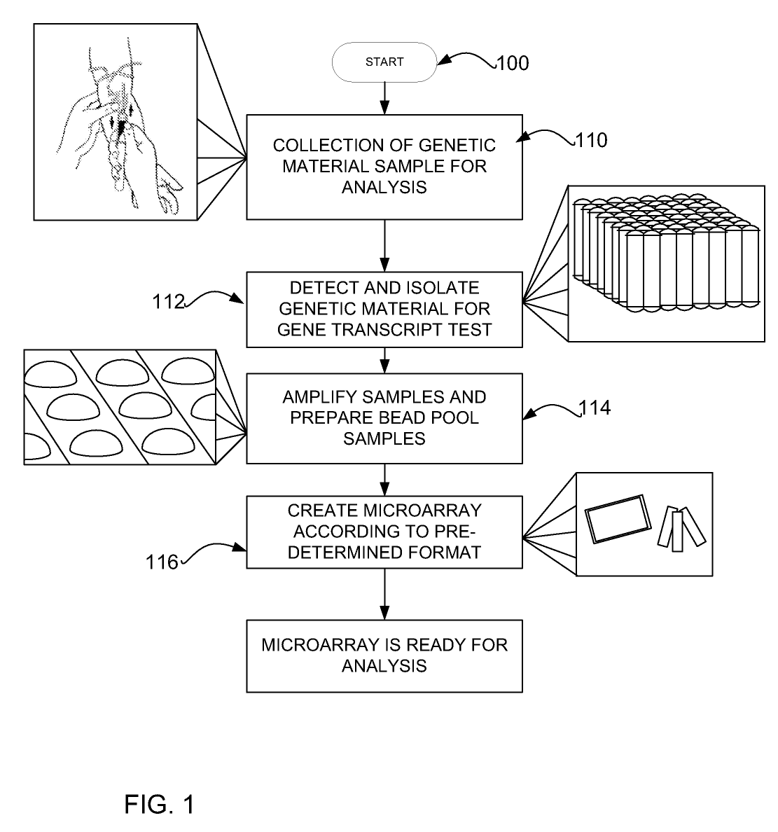

Various embodiments and methods of new processes include the assembly and association of genetic material samples with associated relation to multiple sclerosis, the preparation of microarrays with representative genetic material samples in a pattern best suited for analysis as well as manipulation, and delivery of assimilated and compiled data across a computer network. Various aspects of these embodiments are discussed in FIGS. 1-7 below.

FIG. 1 shows a diagram of an overall method 100 for preparing genetic samples that may be used in a broad-based gene association transcript test for multiple sclerosis according to an embodiment of an invention disclosed herein. The method may typically include drawing a blood sample (or obtaining another source of genetic material) from a patient scheduled for genotyping in step 110. Of course, in order to assimilate a broad-based set of data that encompasses the wide-ranging genetic aspects and symptoms of multiple sclerosis, blood samples are typically drawn from several sources. It should be noted that any tissue suitable for gaining access to genetic material (e.g., DNA and/or RNA) may be used, such as liver tissue. Blood cells are easily collected and easily transported making this source for DNA/RNA efficient and effective. The blood sample may typically be collected using a suitable blood collection device such as blood collection tubes that are available from Paxgene™.

The sample is typically properly tagged and labeled by an anonymous yet traceable patient identification. That is, all measures are taken to comply with the Health Insurance Portability and Accountability Act (HIPAA) such that the blood sample is identifiable but also protected from accidental disclosure of privileged information. At the time of collection, additional demographic information may be stored (e.g., written on a tag, stored in a computer database) with the blood sample. Such demographic information may include a number of different patient characteristics and descriptions, such as age, sex, country of origin, race, specific health issues, occupation, birthplace, current living location, etc.

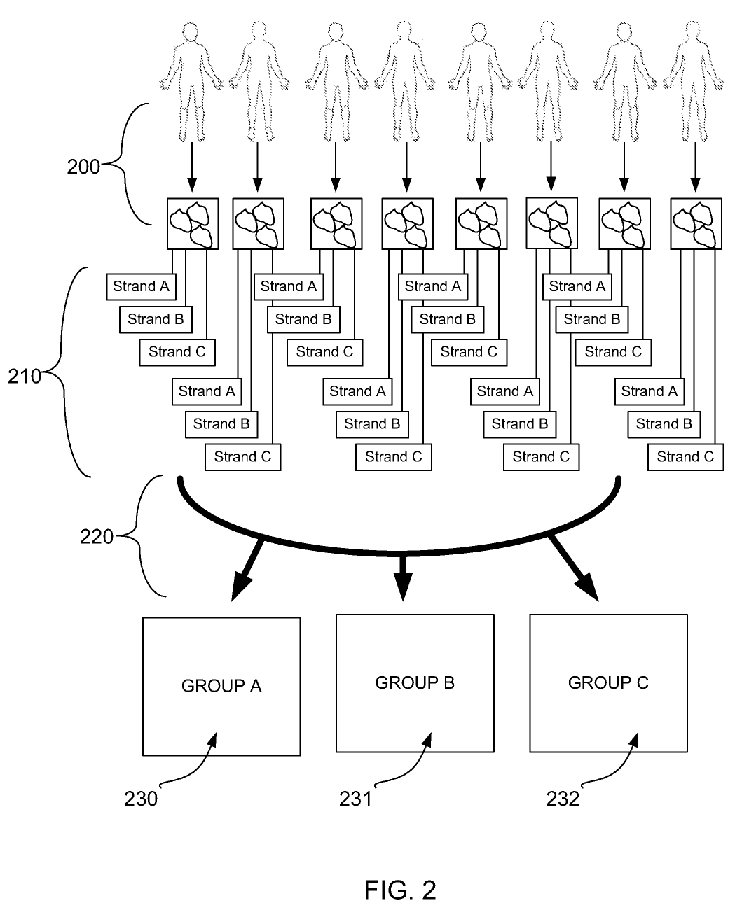

Specific genetic material, such as RNA from the blood sample, may then be detected and isolated in step 112 using an RNA isolation kit such as those that are available from Qiagen™. As mentioned above, RNA isolation may be accomplished at the same physical location as collection or may be accomplished at a remote laboratory after collection. The genetic material isolation process is described in more detail below with respect to FIG. 2.

At step 114, specific sequences in an RNA sample may be amplified using a fluorescence process that may be specific to pre-determined strands of RNA such as available from Illumina™ in a product entitled DASL™. In an alternative embodiment, specific sequences in DNA may also be amplified using a similar fluorescence process that may be specific to pre-determined strands of DNA such as available from Illumina™ in a product entitled Golden Gate™.

The isolation of genetic materials is typically followed by amplification of fluorescently labeled copies that may then be hybridized to specific probes attached to a common substrate, i.e., a microarray. However, the collected and isolated samples may be arranged and analyzed in any manner suitable for analysis. As such, data may be collected and assimilated directly into a computer-based data structure, such as a database, without having to prepare a microarray.

At step 116, the isolated and amplified samples of genetic material may be grouped according to identified sets of strands of genetic material. The groups may be arranged in a specific pattern in bead pools on a microarray according to a predetermined format. Such predetermined formats may include a standard format suitable for individual analysis of all identified genes in isolated RNA/DNA strands. Other predetermined formats may include a side-by-side comparison to one or more control groups of similar genes from control group samples. Other formats may include specific sets of genes suitable for broad-based disease association, multiple sclerosis association, broad-based diagnostics collection, broad-based predictive treatment data sets, or any other association of genes with samples. Once the microarray has been created in a specific pattern, the emergence of patterns and the like may be ready for analysis at step 118. The preparation of such a microarray is described in more detail in U.S. patent application Ser. No. 11/775,660 entitled, “Method and System for Preparing a Microarray for a Disease Association Gene Transcript Test,” assigned to IGD-Intel of Seattle, Wash., which is incorporated by reference. The formats for arranging samples in a microarray typically follow specifics associated with the groupings of blood samples as discussed below with respect to FIG. 2.

FIG. 2 shows a diagrammatic representation of a method for collecting blood samples from several sources and identifying strands of genetic material for grouping according to an embodiment of an invention disclosed herein. In an overview of one method disclosed herein, one may begin the method by collecting a plurality of similar blood samples from a plurality of similar sources, the blood samples suitable for genetic material isolation and analysis. Then, identifiable strands of genetic material in each blood sample may be detected and isolated such that the strands of genetic material identifiable by a gene sequence or nucleotide sequence.

Next, for each blood sample, as an identifiable strand emerges, the samples may be separated into sets of samples with similar identifiable strands and then each set of isolated strand samples of genetic materials may be then grouped into groups of genetic material from each of the plurality of blood samples, such that each group comprises similar identifiable strands of genetic material from each blood sample. Once grouped, each group of genetic material maybe associated with a specific gene relevant to the identifiable strands comprising each group or any other relevant data that may be useful for diagnostics. Aspects of these broad-based steps are discussed below.

In FIG. 2, several different sources of genetic material may typically be used to obtain several different samples of genetic material. This step is represented in the aggregate at step 200 in FIG. 2 and may be associated with the individual step 110 of FIG. 1. As a result, several different and identifiable samples of genetic material may then be processed to detect and isolate specific genetic material for assimilation into an aggregate context. One such process includes RNA isolation.

Specific gene sequences (i.e., nucleotide sequences) may be identified when detecting and isolating strands of genetic material from each sample at step 210. On an aggregate level, each sample may typically have a first strand, such as STRAND A, such that all gene sequences that may be identified as STRAND A may be isolated and the sample separated from all other strands. Likewise, STRAND B for each sample may be also isolated and its respective sample separated. The case is also the same for STRAND C and every other identifiable strand of genetic material in each sample. Although, only 3 specific strands are shown in FIG. 2, it is well understood in the art that the potential strands that may be isolated number in the thousands.

Such isolation processes may comprise the isolating of genetic material based on strands of RNA as identified by a specific gene sequence as described above. Additionally, the isolation of genetic material may be based upon a gene sequence associated with a gene expression indicative of a specific MS trait or even the susceptibility to a specific gene expression of multiple sclerosis, a gene sequence associated with a gene expression indicative of a trait, a gene sequence associated with a gene expression indicative of a phenotype, and/or a gene sequence associated with a gene expression indicative of a genotype.

With all groups of strands detected, isolated, and identified, each set of strands (i.e., all samples with STRAND A isolations) across all samples may be grouped together for additional association and analysis at step 220. As such, all expressions of STRAND A may be grouped into GROUP A 230, all expressions of STRAND B may be grouped into GROUP B 231 and all expressions of STRAND C may be grouped into GROUP C 232. Such grouping allows for the assimilation of data on an aggregate level based on various gene expressions as compared to a number of aggregate level aspects of assimilated data. Specifically, demographic information about the source of a sample may be associated with each sample.

Additionally, aggregating information associated with each blood sample may be accomplished through the groupings of similar strands. Such aggregating includes associating a blood sample exhibiting an expression of a gene sequence indicative of a first MS trait or susceptibility to the first MS trait with the demographic information about the blood sample, associating a blood sample exhibiting an expression of a gene sequence indicative of a first MS trait with another blood sample exhibiting an expression of a gene sequence indicative of the first MS trait, associating a blood sample exhibiting an expression of a gene sequence indicative of a first MS trait with a blood sample exhibiting an expression of a gene sequence indicative of a second MS trait, associating a blood sample exhibiting an expression of a gene sequence indicative of a first MS trait with a treatment associated with the first MS trait, and associating a blood sample exhibiting an expression of a gene sequence indicative of a first MS trait with a specific polymorphism.

With any number of associations in place from the groupings, statistical data from the aggregated blood samples based on associations of one blood sample with another may be extrapolated. Such statistical data may include expression rates, inter-related expression rates, etc, of many different multiple sclerosis traits, symptoms, and/or gene expressions.

Application of this unique set of probes will offer a low cost genomic assessment of an individual's state of health through a new and useful clinical diagnostic with regard to multiple sclerosis and/or a person's susceptibility to multiple sclerosis. Additionally, adding or deleting probes that relate to a given MS trait, as new information is presented in peer-reviewed literature may further enhance the benefits of the clinical diagnostic. Adding probe content as information expands is a planned future course of action, as will be appreciated by others in the art. Further yet, the clinical diagnostic may be expanded such that components may be tested as separate, and/or all inclusive tests that address different diseases, job-related concerns, or lifestyle concerns.

Information that may now be gleaned from the groupings of sets of genetic material may be aggregated into in a computer readable medium accessible by a server computer, e.g., a database. Then, such data may be accessed by any connected client computer such that information is provided from the aggregated data to a client computer upon a request from the client computer to the server computer.

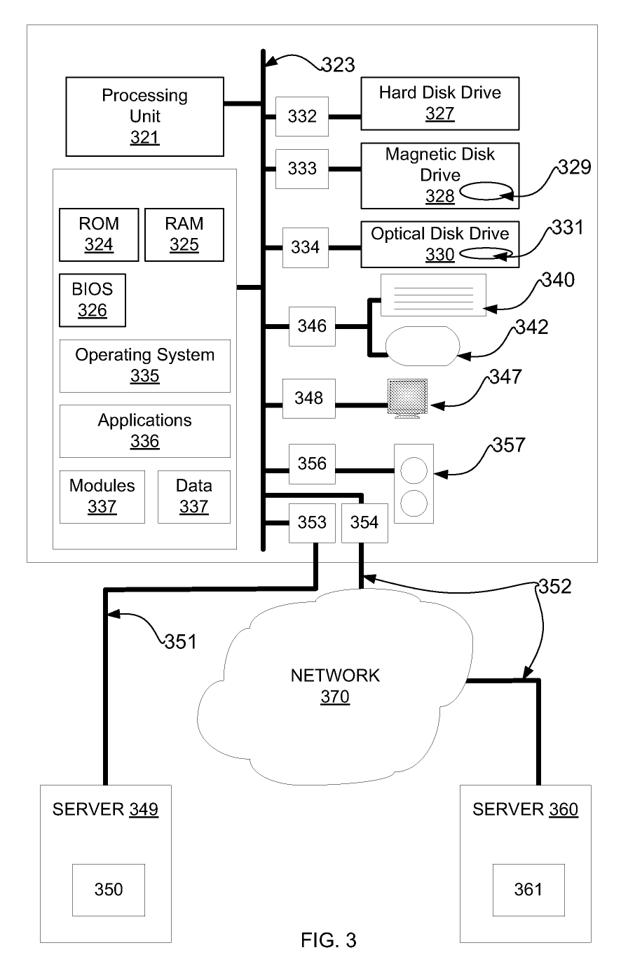

FIG. 3 is a diagrammatic representation of a suitable computing environment in which some aspects of a broad-based gene association transcript test for multiple sclerosis may be practiced according to an embodiment of an invention disclosed herein. With reference to FIG. 3, an exemplary system for implementing the invention includes a general purpose computing device in the form of a conventional personal computer 320, including a processing unit 321, a system memory 322, and a system bus 323 that couples various system components including the system memory to the processing unit 321. The system bus 323 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The system memory includes read only memory (ROM) 324 and random access memory (RAM) 325. A basic input/output system (BIOS) 326, containing the basic routines that help to transfer information between elements within the personal computer 320, such as during start-up, is stored in ROM 324. The personal computer 320 further includes a hard disk drive 327 for reading from and writing to a hard disk, not shown, a magnetic disk drive 328 for reading from or writing to a removable magnetic disk 329, and an optical disk drive 330 for reading from or writing to a removable optical disk 331 such as a CD ROM or other optical media. The hard disk drive 327, magnetic disk drive 328, and optical disk drive 330 are connected to the system bus 323 by a hard disk drive interface 332, a magnetic disk drive interface 333, and an optical drive interface 334, respectively. The drives and their associated computer-readable media provide nonvolatile storage of computer readable instructions, data structures, program modules and other data for the personal computer 320. Although the exemplary environment described herein employs a hard disk, a removable magnetic disk 329 and a removable optical disk 331, it should be appreciated by those skilled in the art that other types of computer-readable media which can store data that is accessible by a computer, such as magnetic cassettes, flash memory cards, digital versatile disks, Bernoulli cartridges, random access memories (RAMs), read only memories (ROM), and the like, may also be used in the exemplary operating environment.

A number of program modules may be stored on the hard disk, magnetic disk 329, optical disk 331, ROM 324 or RAM 325, including an operating system 335, one or more application programs 336, other program modules 337, and program data 338. A user may enter commands and information into the personal computer 320 through input devices such as a keyboard 340 and pointing device 342. Other input devices (not shown) may include a microphone, joystick, game pad, satellite dish, scanner, or the like. These and other input devices are often connected to the processing unit 321 through a serial port interface 346 that is coupled to the system bus, but may be connected by other interfaces, such as a parallel port, game port or a universal serial bus (USB). A monitor 347 or other type of display device is also connected to the system bus 323 via an interface, such as a video adapter 348. One or more speakers 357 are also connected to the system bus 323 via an interface, such as an audio adapter 356. In addition to the monitor and speakers, personal computers typically include other peripheral output devices (not shown), such as printers.

The personal computer 320 operates in a networked environment using logical connections to one or more remote computers, such as remote computers 349 and 360. Each remote computer 349 or 360 may be another personal computer, a server, a router, a network PC, a peer device or other common network node, and typically includes many or all of the elements described above relative to the personal computer 320, although only a memory storage device 350 or 361 has been illustrated in FIG. 3. The logical connections depicted in FIG. 3 include a local area network (LAN) 351 and a wide area network (WAN) 352. Such networking environments are commonplace in offices, enterprise-wide computer networks, intranets and the Internet. As depicted in FIG. 3, the remote computer 360 communicates with the personal computer 320 via the local area network 351. The remote computer 349 communicates with the personal computer 320 via the wide area network 352.

When used in a LAN networking environment, the personal computer 320 is connected to the local network 351 through a network interface or adapter 353. When used in a WAN networking environment, the personal computer 320 typically includes a modem 354 or other means for establishing communications over the wide area network 352, such as the Internet. The modem 354, which may be internal or external, is connected to the system bus 323 via the serial port interface 346. In a networked environment, program modules depicted relative to the personal computer 320, or portions thereof, may be stored in the remote memory storage device. It will be appreciated that the network connections shown are exemplary and other means of establishing a communications link between the computers may be used.

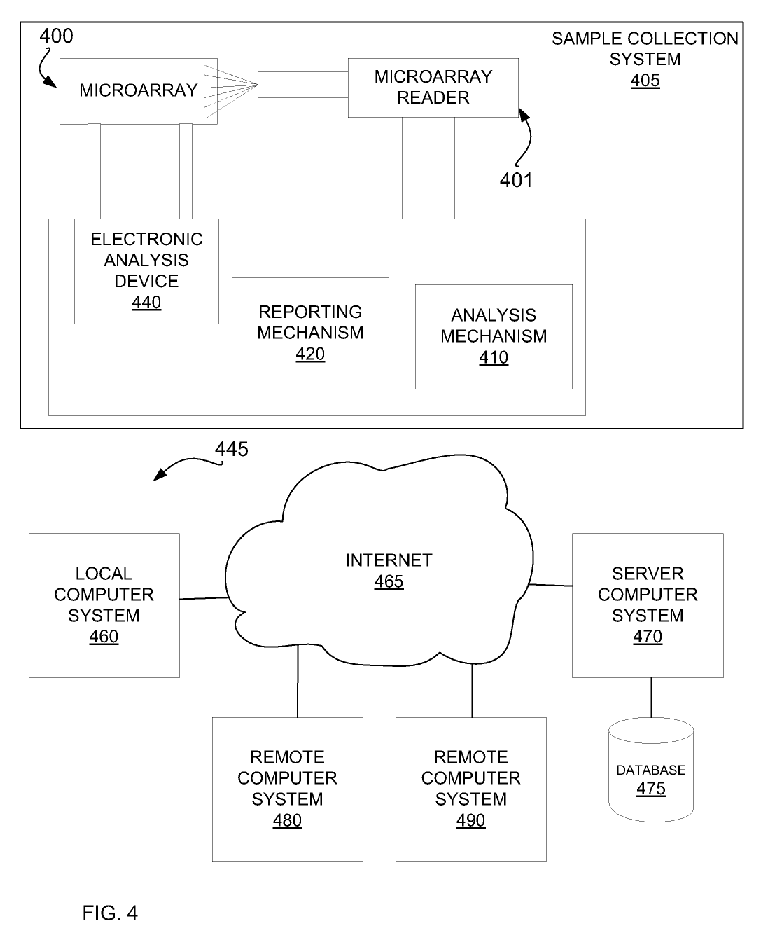

FIG. 4 shows a diagrammatic representation of a method and system for establishing a broad-based gene association transcript test for multiple sclerosis according to an embodiment of an invention disclosed herein. In this embodiment, a microarray 400 may be characterized by an arrangement of different identified gene expressions related to MS based upon an association with many different samples and many different sample sources. Several other arrangements of data exist as other embodiments as well. As such, depending on the known arrangement of samples, specific patterns of the presence of phenotypes or lack thereof determine the type of information to be garnered from each prepared microarray 400. As a result of this embodiment, specific patterns emerge indicating a likelihood of occurrence of a SNPs, insertions, or deletions in various regions, and, likewise, inter-related data that may associate various gene expressions and other data related to MS.

Such patterns may be read by a microarray reader 401. The microarray reading device typically includes a microarray station 402 operable to view a microarray 400. As briefly discussed above, a typical microarray 400 will include a plurality of deposit wells suitable for hosting samples of genetic material. The wells disposed on a substrate may be arranged such that each row is suited for hybridizing a genetic material sample such that a unique gene expression may be identified (i.e., one gene per row). Further, each column is suited for having each sample in each row in the column be associated with a single source of genetic material (i.e., one person per column).

The microarray reader 401 may also typically include an analysis mechanism 410 operable to analyze a pattern displayed on the microarray 400 and a reporting mechanism 420 operable to deliver a report of the analysis. The microarray reader 401 may also have an electronic microarray assessment apparatus 440 operable to determine a pattern of gene expression from a series of electrical pulses sent to and received from the stationed microarray 400.

Microarrays 400 are quite useful is mapping or “expressing” data about the makeup of the genetic material disposed thereon. Applications of these microarrays 400 include the following. Messenger RNA or Gene Expression Profiling—monitoring expression levels for thousands of genes simultaneously is relevant to many areas of biology and medicine, such as studying treatments, mutations, and developmental stages. For example, microarrays 400 can be used to identify diseased genes or mutated genes by comparing gene expression in non-mutated and normal cells. Other uses for microarrays 400 are known and/or contemplated but not discussed herein for brevity.

With such a microarray 400 available for analysis and coupled with several other additional prepared microarrays, broad-based data about the occurrence or absence of MS traits and/or specific gene sequences begins to emerge. The microarray 400 may be scanned and intensity data extracted to associate presence/absence of genetic material in the original sample. This data may be assimilated in a large database of information together with additional information such as diagnosis and treatment information, to provide a multitude of information about a large number of data sets. As the data is assimilated, a comprehensive literature search offering substantiated associations of MS traits with gene expression alterations may be provided. The data are rendered anonymous and uploaded into a central repository that allows cross-sample comparison and ultimately, earlier detection of multiple sclerosis.

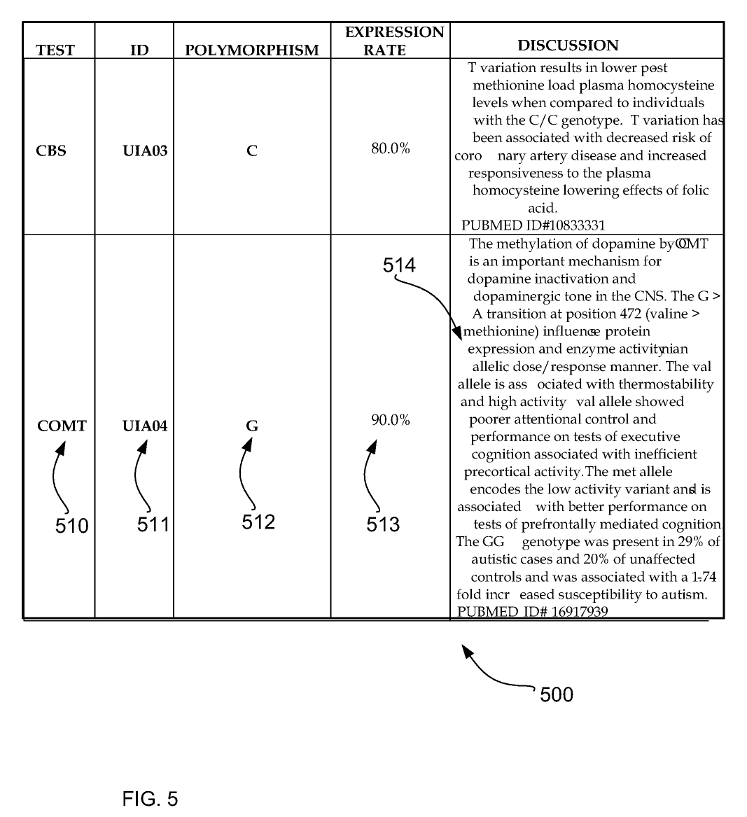

FIG. 5 shows a typical arrangement of data that may be associated in a database of information derived from a broad-based gene association transcript test for multiple sclerosis according to an embodiment of an invention disclosed herein. The data associated with the portions of genetic material stemming from traceable samples may be arranged in a data structure 500 according to FIG. 5. In FIG. 5, the data structure may associate a specific test 510, an ID 511, a polymorphism 512, an expression rate 513, and a discussion 514.

The specific test 410 may typically comprise a known set of nucleotide sequences in which one should examine to determine the presence or non-existence of specific genetic disease or genetic disorder. Based on the polymorphism 412, and ratio 413, the interpretation 414 will indicate the possibilities for diagnosis, or suggest treatment for a specific illness.

The ID 411 may typically comprise the unique identification measure that removes individual identity and replaces it with associative phenotypic characteristics.

The Polymorphism 412 may typically refer to the specific nucleotide that is present for the sample analyzed and may be associated with the presence of a disease. That is, in the specific nucleotide sequence identified in the polymorphism 412, relates to the proportion of analyzed genomic sequences that result from the processing of the test for each individual.

Finally, the data structure may also include a discussion 414 that is obtained from clinically relevant understanding from sources of peer reviewed literature and published clinical studies.

With at least some of these data sets in a data structure, a broad-based gene association transcript test for multiple sclerosis be realized. Such a data structure may be characterized by a first tangible (i.e., fixed in some tangible medium) data set operable to store a gene expression isolated from genetic material from a specific source, the gene expression associated with a first MS trait, a second tangible data set operable to store an identification of the source and associated with the first tangible data set, a third data set associated with a specific demographic characteristic linked to the first MS trait and a fourth tangible data set operable to store at least one other association with a second MS trait, the second MS trait associated with a second gene expression.

Additional data sets may include a fifth tangible data set operable to store an identification of a specific test associated with the first MS trait, a sixth tangible data set operable to store an expression rate associated with the first MS trait and associated with the first gene expression, and a seventh tangible data set operable to store a discussion associated with the first MS trait and associated with the first gene expression. Such a data structure may be realized in a fixed computer-readable medium, such as a database, or may be fixed to another medium such as a substrate hosting a microarray of genetic samples.

Such a data structure allows for an assessment and/or analysis of all newly collected genealogical data. For example, if demographics data about the source of the sample was collected at the same time that the sample was collected, the demographics data may also be associated with the expression of specific MS trait by associating the demographics data with the portions of each sample exhibiting an expression for such a MS trait. Then, with these data associations in place within the data structure, such associative data may be extrapolated that encompasses a first MS trait associated with a portion of a sample with the demographic information about the source of the sample. In the aggregate, specific trends about demographic data and specific MS traits may be garnered.

As another example, additional trend data may be garnered by associating a portion of a sample from a first source exhibiting the specific gene expression indicative of a first MS trait with a portion of a sample from the first source exhibiting the specific gene expression indicative of a second MS trait. Then, with these associations in place additional trend data may be garnered by extrapolating associative data encompassing a portion of a sample from a first source exhibiting the specific gene expression indicative of a first MS trait with a portion of a sample from the first source exhibiting the specific gene expression indicative of a second MS trait. Similarly, such trend data may be garnered by associating specific polymorphisms with specific portions exhibiting such nucleotide sequences associated with the polymorphisms.

Additional information about multiple sclerosis associations may be garnered by associating the portions from the first sample respectively exhibiting specific gene expressions associated with the first and second MS trait with a portion of a sample from a second source exhibiting the specific gene expressions associated with either the first or the second MS trait. With these associations, one may extrapolate associative data regarding a portion of a sample from a first source exhibiting the specific gene expression indicative of a first MS trait, a portion of a sample from the first source exhibiting the specific gene expression indicative of a second MS trait, and a portion of a sample from a second source exhibiting the specific gene expressions associated with either the first or the second MS trait in an effort to yield additional trend data.

As yet another example, treatment data may be expressed by associating a portion of a sample from a first source exhibiting the specific gene expression indicative of a first MS trait with a treatment linked to the first MS trait. Further, such treatment data may also be extrapolated from such associative that encompasses a portion of a sample from a first source exhibiting the specific gene expression indicative of a first MS trait with a treatment linked to the first MS trait.

A specific combination of nucleic acid sequences taken from isolated regions of the human genome may be reflected as custom content on a platform independent gene expression microarray for multiple sclerosis. A complete list of nucleic acid sequences form the elements analyzed within this human genome examination may form the basic nature of a gene transcript test for multiple sclerosis, which is typically intended for clinical use in effectively detecting transcribed alterations in the genetic code that have a documented relationship with one or more aspects of multiple sclerosis, association with therapeutic response, and/or treatment for one or more multiple sclerosis symptoms. The content of the test may assess RNA through quantitative (measurement and assessment of transcript present within the tissue) and qualitative (measurement of genomic regions) means.

This nucleic acid array may be comprised of probe sequences isolated to detect regions within a given gene that most effectively indicate expression levels and that represent polymorphic sections indicating which sequence from the genome an individual is actually expressing. The nucleic acid sequences deemed present in the amplified portions of a sample isolated from standard blood draw and/or genetically mutated tissue, may be detected by hybridizing the amplified portions to the array and analyzing a hybridization pattern resulting from the hybridization.

Association of test results with claims and assessments of clinical relevance may be assimilated and documented as conclusions formed through a comprehensive compilation of peer-reviewed literature (or other periodic update). Ongoing modifications to these claims and assessments may be performed through quarterly protocol assessment and maintenance of a peer-to-peer physician support network supported through existing and impending corporate associations.

Paper reporting of the test results may indicate the outcome from a subset of 1 to 50 genetic sequences related to various MS traits. Additional reporting for several other sequences may be made available through alternative measures. These measures may enable physicians to access their patient's information relative to all other patients having ordered the test through a variety of associative clustering methods (hierarchical, divisive, and associative). The concept of creating real-time genotype/phenotype association accessible to physician-to-physician networks may be further promoted as a desired goal. Physicians will be able to analyze their own patient's data relative to all other data existing individuals who have had the test performed.

Examples of polymorphisms assessed may be single nucleotide polymorphisms (SNPs), deletions, and/or deletion insertion sequences. Further, the polymorphisms predicted to be present in the amplified portions may already be determined. Further yet, the nucleic acid sample may be genomic DNA, cDNA, cRNA, RNA, total RNA or mRNA. With these variations, the SNP, deletion, or insertion may be associated with a multiple sclerosis, the efficacy of a drug, and/or associated with predisposition towards/against development of aforementioned ailment(s). Typically, output data may be packaged in a computer-readable medium (e.g., a CD or DVD) and delivered to a customer, such as a subscribing physician.

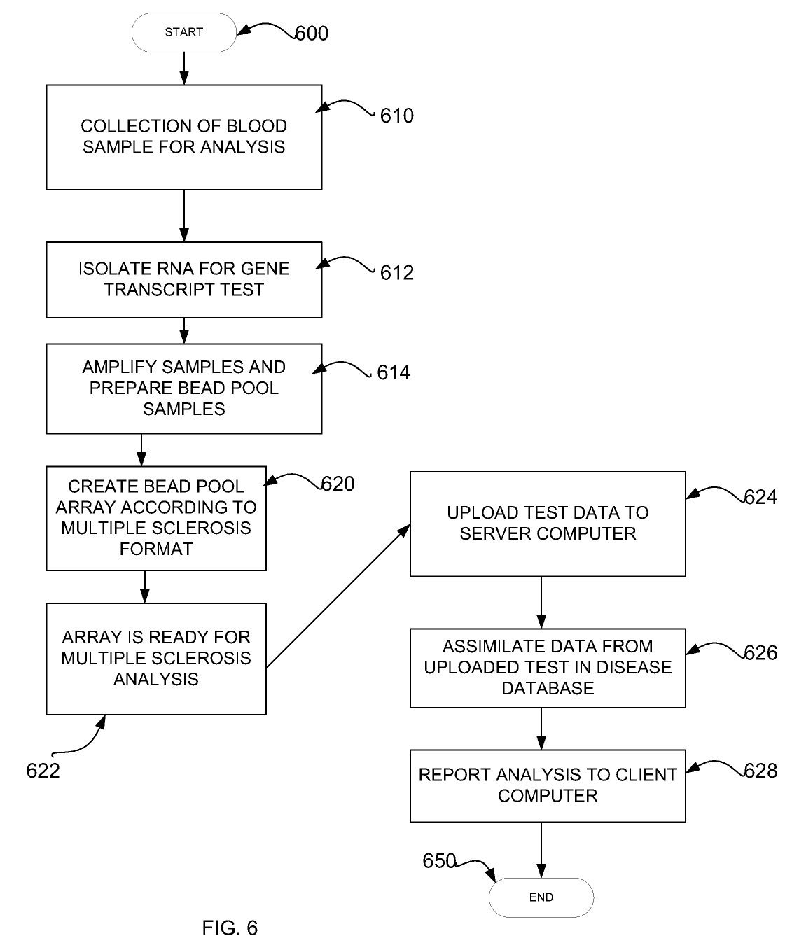

FIG. 6 is a flow chart of a method for collecting genetic material and preparing the collection for assessment in a broad-based gene association transcript test for multiple sclerosis according to an embodiment of an invention disclosed herein. In this aspect of the overall method, specific data may be garnered from analysis of a prepared microarray and then uploaded to a server computer to be assimilated into a database of multiple sclerosis gene expression data.

The method begins at step 600 and proceeds to step 610 where a collection of blood samples for analysis is gathered. A process for collecting blood samples was described above with respect to FIGS. 1 and 2. At step 612, also as described above, RNA from each blood sample may be isolated for a gene transcript test and then each isolated sample amplified in preparation for depositing into the bead pools onto a microarray in step 614.

In this method, the bead pools are arranged according to a specific format suitable for multiple sclerosis analysis. As described above, many formats are possible and are typically unique to the specific aspects of the particular disease in which a prepared microarray is to be used. As such, at step 620, a microarray is prepared according to a multiple sclerosis format and is then ready for multiple sclerosis analysis at step 622. Such an analysis typically yields data that may yield specific information about the sample as well as provide additional data about multiple sclerosis that may be assimilated into a database of such information.

Thus, once data has been garnered from an analysis of a multiple sclerosis format microarray, such test data may be uploaded to a server computer hosting a database of multiple sclerosis information at step 624. Such uploaded data may or may not be worthy of inclusion into the database. For example, the data may fall outside of standard deviations and therefore not trustworthy for using within the assimilated data of the database. If this is determined, the uploaded data is typically discarded and the user who uploaded the data is notified. However, if the data is determined to be valid i.e. worthy of inclusion into the database, then the data is assimilated into the multiple sclerosis database at step 626. Based upon an analysis of the newly uploaded data and the existing assimilated data and analysis, which may take into account all aggregated data within the database, may be generated and delivered to a client computer. This analysis, reported at step 628, may typically provide any number of details about associations of genes exhibiting particular expressions related to multiple sclerosis. The process ends at step 650.

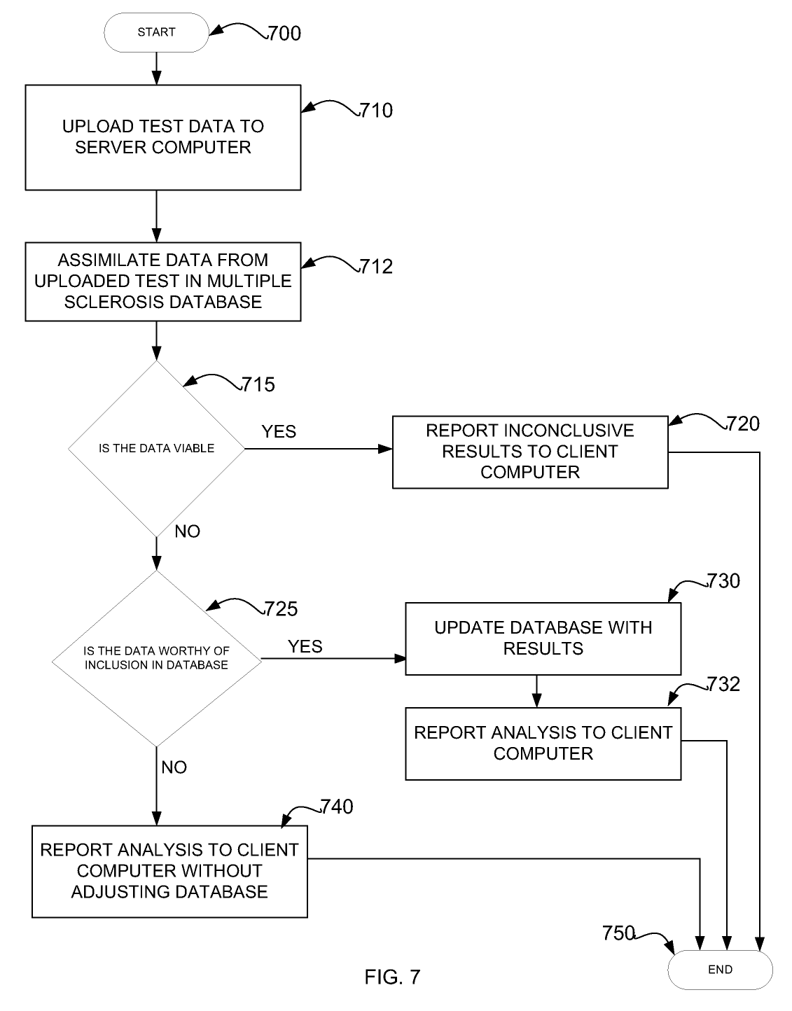

FIG. 7 is a flow chart of a method for diagnosing and/or screening a patient for potential genetic expressions of MS traits according to an embodiment of an invention disclosed herein. The method depicted here in FIG. 7 presumes that genetic samples from at least one source have been collected and prepared for assimilation. As such, in an overview of one computer-related method and/or one set of computer executable instructions fixed in a computer-readable medium depicted in FIG. 7, one may collect genealogical data about a person and store the collected data in a data set at a client computer, wherein the genealogical data includes a plurality of genetic sequences. Then, the data set may be transmitted to a server computer that is communicatively coupled to the client computer. The server computer may assess the data set to determine an assessment of at least one genetic sequence that is associated with a specific MS trait. Once assessed, the server computer may return the assessment to the client computer.

Thus, a step 710, the assimilated genealogical test data may be transmitted (i.e., uploaded) to a server computer that hosts various database and analysis programs for a broad-based gene association transcript test for multiple sclerosis. The genealogical data may be collected from an analysis of a microarray that includes genetic material samples from the person. The data set may have specific association embedded therein in including associations between the genealogical data and information about the source of the genealogical data as well as associations between specific isolations of the genealogical data and a corresponding MS trait.

At step 712, the uploaded test data may be assimilated into a database of aggregately associated MS analysis data. At step 715, the server computer may analyze the uploaded data to determine if the data is valid. Valid data may be verified by a statistical analysis of the data presented. Results that fall outside of one or two standard deviations from all previously assimilated data may be deemed to be invalid. Invalid data may be discarded and not assimilated into the database. Invalid results may then be reported to the client at step 720.

If, however, the data sets are determined to be valid, a second assessment of the data sets occurs at step 725. Thus, the data set is assessed as to its worthiness for inclusion in the database. If the data is duplicative of other data already assimilated, then no need exists for its inclusion. Further, if all relevant associations and conclusion based on an analysis yields no new information, again, the data may simply be discarded without assimilation into the database. An analysis is reported to the client without assimilating the data at step 740. If the data is particularly useful, the database may be updated at step 730 and the client notified at step 732. The method of FIG. 7 ends at step 750.

Further, as the database is updated with valid and worthy data, other connected client computers may also be notified of the changes to the database. This allows for other physicians to see new results and likewise review such results for use with their own patients and diagnostics. Further yet, the entire method described above may also be applied in the context of assessing a person's susceptibility to multiple sclerosis.

While the subject matter discussed herein is susceptible to various modifications and alternative constructions, certain illustrated embodiments thereof are shown in the drawings and have been described above in detail. It should be understood, however, that there is no intention to limit the claims to the specific forms disclosed, but on the contrary, the intention is to cover all modifications, alternative constructions, and equivalents falling within the spirit and scope of the claims.

What is claimed is:

1. A method for assessing a genetic expression of multiple sclerosis by assembling gene transcript test data from a plurality of genetic material sources, the method comprising:
obtaining a sample of genetic material from a person exhibiting a potential for multiple sclerosis;
for each sample, isolating portions of each sample such that each isolated portion exhibits a specific gene expression associated with one of a plurality of multiple sclerosis symptoms, each isolated portion corresponding uniquely with an associated symptom;
disposing each portion on a microarray in a pattern suitable for multiple sclerosis genotyping;
aggregating data by assimilating one or more patterns of gene expression on the microarray in comparison into a data structure of a plurality of sets of data about multiple sclerosis genetic expression from a plurality of genetic material sources; and
assessing the aggregated data for the expression of multiple sclerosis traits.

2. The method of claim 1, further comprising associating demographic data about the source of each sample with each portion of each sample.

3. The method of claim 2, further comprising extrapolating associative data from the data structure, the associative data encompassing a first multiple sclerosis trait associated with a portion of a sample with the demographic information about the source of the sample.

4. The method of claim 1, wherein the assessing comprises associating a portion of a sample from a first source exhibiting the specific gene expression indicative of a first multiple sclerosis trait with a portion of a sample from the first source exhibiting the specific gene expression indicative of a second multiple sclerosis trait.

5. The method of claim 1, wherein the assessing comprises associating the portions from the first sample respectively exhibiting specific gene expressions associated with the first and second multiple sclerosis trait with a portion of a sample from a second source exhibiting the specific gene expressions associated with either the first or the second multiple sclerosis trait.

6. The method of claim 1, wherein the assessing comprises associating a portion of a sample from a first source exhibiting the specific gene expression indicative of a first multiple sclerosis trait with a treatment linked to the first multiple sclerosis trait.

7. The method of claim 1, wherein the assessing comprises associating a portion of a sample from a first source exhibiting the specific gene expression indicative of a first multiple sclerosis trait with a specific polymorphism.

8. The method of claim 1, further comprising:
collecting genealogical data about the person and storing the collected data in a data set at a client computer, the genealogical data including a plurality of genetic sequences;
transmitting the data set to a server computer that is communicatively coupled to the client computer;
assessing the data set to determine at least one genetic sequence that is associated with a specific multiple sclerosis trait; and
returning the assessment to the client computer.

9. The method of claim 8 wherein the genealogical data is collected from an analysis of a microarray that includes genetic material samples from the person.

10. The method of claim 8 wherein the collecting the genealogical data into the data set further comprises:
associating the genealogical data with information about the source of the genealogical data; and
associating specific isolations of the genealogical data with a corresponding multiple sclerosis trait.

11. The computer-related method of claim 8 wherein assessing the data set further comprises:
comparing the data set to a database of information assembled from many other similar data sets that are stored in the data structure;
identifying specific genetic sequence in the transmitted data set that correspond to one or more multiple sclerosis trait;
identifying specific genetic sequences in the transmitted data set that corresponds to susceptibility to a multiple sclerosis trait; and
determining a rate of expression for each identified genetic sequence.

12. The method of claim 8, further comprising:
determining if the data from the transmitted data set is valid; and
updating the data structure with the newly transmitted data set.

13. The method of claim 12 further comprising:
notifying the client computer that the data set is valid; and
notifying the client computer that the data structure is updated with the transmitted data set.

14. The method of claim 12 further comprising notifying at least one other client computer that the database has been updated with a new data set.

15. A system for assessing genealogical data for susceptibility to multiple sclerosis, the system comprising:
a client computer operable to store collected genealogical data about a person in a data set in a data store, the collected genealogical data including a plurality of genetic sequences;
a data transmission device coupled to the client computer and operable to transmit the data set to a server computer that is communicatively coupled to the client computer;
an assessment program module executing at the server computer, the assessment program operable to assess the data set to determine an assessment at least one genetic sequence that is associated with a specific multiple sclerosis trait; and
a reporting program module executing at the server computer, the reporting program operable to return the assessment to the client computer.

16. The system of claim 15, further comprising a validation program module executing on the server computer, the validation program module operable to determine if the newly generated assessment based upon the transmitted data set is valid.

17. The system of claim 16, further comprising an update program module executing on the server computer, the update program module operable to update a database of assessments with the newly generated assessment based upon the transmitted data set if the data set is determined to be valid.

18. The system of claim 17, further comprising reporting program module executing on the server computer, the reporting program module operable to report a valid update to the database to a plurality of other communicatively coupled client computers.

19. The system of claim 15, further comprising a sample collection system operable to collect genealogical data by analyzing a microarray of genetic material, the sample collection system coupled to the client computer.

20. A computer-related method for a gene association gene transcript test for multiple sclerosis, the method comprising:
collecting genealogical data about a person and storing the collected data in a data set at a client computer, wherein the genealogical data includes a plurality of genetic sequences;
transmitting the data set to a server computer that is communicatively coupled to the client computer;
assessing the data set at the server computer to determine an assessment of at least one genetic sequence that is associated with a specific MS trait; and
returning the assessment to the client computer.
